(19) United States
(12) Patent Application Publication
(10) Pub. No.: US 2006/0178721 A1
(43) Pub. Date: Aug. 10, 2006

(54) Stent delivery balloon catheter having improved stent retention

(75) Inventors: Jonathan P. Durcan, Temecula, CA (US); Kerry J. Williams, Temecula, CA (US)

Correspondence Address: FULWIDER PATTON, 6060 CENTER DRIVE, 10TH FLOOR, LOS ANGELES, CA, 90045, US

(73) Assignee: ADVANCED CARDIOVASCULAR SYSTEMS, INC.

(21) Appl. No.: 11/057,022

(22) Filed: Feb. 10, 2005

(51) Int. Cl. A61F 2/06

(52) U.S. Cl. 623/1.11

(57) ABSTRACT

A stent delivery balloon catheter having improved stent retention. In one embodiment, a stent mounted on the balloon catheter is embedded in an outer surface of an elastomeric sleeve on the catheter balloon such that the stent forms an imprint in the outer surface of the sleeve. One aspect of the invention is directed to a method of mounting a stent on the balloon catheter. The stent is securely mounted on the balloon due to the interference with the imprinted sleeve.

### BACKGROUND OF THE INVENTION

This invention relates generally to catheters, and particularly intravascular catheters for use in the delivery of stents.

In percutaneous transluminal coronary angioplasty (PTCA) procedures a guiding catheter is advanced in the patient's vasculature until the distal tip of the guiding catheter is seated in the ostium of a desired coronary artery. A guidewire is first advanced out of the distal end of the guiding catheter into the patient's coronary artery until the distal end of the guidewire crosses a lesion to be dilated. A dilatation catheter, having an inflatable balloon on the distal portion thereof, is advanced into the patient's coronary anatomy over the previously introduced guidewire until the balloon of the dilatation catheter is properly positioned across the lesion. Once properly positioned, the dilatation balloon is inflated with inflation fluid one or more times to a predetermined size at relatively high pressures so that the stenosis is compressed against the arterial wall and the wall expanded to open up the vascular passageway. Generally, the inflated diameter of the balloon is approximately the same diameter as the native diameter of the body lumen being dilated so as to complete the dilatation but not overexpand the artery wall. After the balloon is finally deflated, blood flow resumes through the dilated artery and the dilatation catheter and the guidewire can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate of angioplasty alone and to strengthen the dilated area, physicians now normally implant an intravascular prosthesis, generally called a stent, inside the artery at the site of the lesion. Stents may also be used to repair vessels having an intimal flap or dissection or to generally strengthen a weakened section of a vessel or to maintain its patency. Stents are usually delivered to a desired location within a coronary artery in a contracted condition on a balloon of a catheter which is similar in many respects to a balloon angioplasty catheter, and expanded within the patient's artery to a larger diameter by expansion of the balloon. The balloon is deflated to remove the catheter and the stent left in place within the artery at the site of the dilated lesion. See for example, U.S. Pat. No. 5,507,768 (Lau et al.) and U.S. Pat. No. 5,458,615 (Klemm et al.), which are incorporated herein by reference.

One difficulty has been retention of the stent on the catheter balloon during delivery and deployment of the stent in a patient's body lumen. If the stent is dislodged from or moved relative to the balloon the system will not correctly implant the stent into the body lumen. However, the stent can't be so strongly fixed to the balloon that it inhibits expansion of the balloon and/or release of the stent once the balloon is positioned at the desired location. Additionally, the stent mounting procedure must not damage a drug or drug delivery matrix on the stent delivery system. It would be a significant advance to provide a catheter balloon having improved stent retention, and without inhibiting balloon or catheter function. The present invention satisfies these and other needs

### SUMMARY OF THE INVENTION

The invention is directed to a stent delivery balloon catheter having improved stent retention. In one embodiment, a stent mounted on the balloon catheter is embedded in an outer surface of an elastomeric sleeve on the catheter balloon such that the stent forms an imprint in the outer surface of the sleeve. The stent is securely mounted on the balloon due to the interference with the imprinted sleeve.

The stent delivery balloon catheter generally comprises an elongated shaft having an inflation lumen and a guidewire lumen, a balloon on a distal shaft section having an interior in fluid communication with the inflation lumen, and a stent releaseably mounted on the balloon for delivery and deployment within a patient's body lumen. The balloon typically has a folded noninflated configuration with wings wrapped around the circumference of the balloon. The stent typically comprises an open-walled body of stent struts with gaps between adjacent struts. The stent delivery system has a therapeutic agent typically carried by the stent. For example, in one embodiment, the stent has a drug delivery coating on a surface of the stent. The stent can alternatively carry the therapeutic agent by a variety of suitable methods as are well known in the art, including forming the stent body or an outer cover of a substance containing the therapeutic agent, or providing the stent with a reservoir containing the therapeutic agent. The stent and/or drug delivery coating are biostable or bioabsorbable.

An elastomeric sleeve is on an outer surface of the balloon, between the stent and balloon. At least a section of the elastomeric sleeve is bonded to the balloon and/or to the catheter shaft, for example by an adhesive or heat-fusion bond. During mounting of the stent on the sleeve, the elastomeric material of the sleeve is caused to flow, and as a result, an imprint of the stent is formed in the outer surface of the sleeve. The imprint is present in the sleeve in a noninflated configuration, an inflated configuration, and a deflated configuration after the inflated balloon is deflated to radially collapse the balloon and sleeve away from the expanded stent. Thus, the imprint of the stent in the sleeve is permanent absent a further reflowing and remolding of the sleeve.

The stent fits within the imprint in the sleeve on the noninflated balloon, with portions of the sleeve having an outer diameter which is at least larger than the inner diameter of the nonexpanded stent mounted on the balloon, thereby improving stent retention. Specifically, the portions of the sleeve which protrude within the stent gaps provide mechanical interference between the stent and the sleeve, thus preventing or inhibiting longitudinal movement of the stent relative to the sleeve during delivery and deployment of the stent, without forming a bond or other adhesive connection between the stent and sleeve. Additionally, the sleeve is typically a softer material than that used to form the balloon thereby providing increased frictional forces between the stent and the sleeve.

Unlike a folded balloon, the sleeve radially expands from a nonfolded noninflated configuration without unfolding. As the sleeve begins to radially expand with the balloon, the stent remains embedded in the imprint and unlikely to move longitudinally relative to the sleeve. Typically, at some point during inflation of the balloon, the deformation of the stent is such that the stent no longer fits within the imprint.

The sleeve is formed of an elastomeric polymer. Suitable thermoplastic elastomers (TPE) have a recoverable strain greater than approximately 300% with little permanent set. Preferably, the material has a recoverable strain of about 600%, with less than 10% permanent set (tension set). The material also has a low glass transition temperature as discussed in more detail below. The materials that meet these criteria include polyurethane, latex and styrenic block copolymers. One preferred material is a blend of 75% Tecoflex 80A (a polyurethane copolymer) and 25% Vector 7400 (a styrene-butadiene-styrene (SBS) block copolymer).

The elastomeric material preferably has a glass transition temperature which is relatively low. For example, in one embodiment the glass transition temperature of the sleeve material is below room temperature. Thus, in a preferred embodiment, the glass transition temperature of the elastomeric material forming the sleeve is less than the glass transition temperature of the polymeric material forming the balloon. Due to the low glass transition temperature of the elastomeric material, the material is caused to reflow at relatively low temperatures during mounting of the stent thereon, and preferably at temperatures and inflation pressures which are too low to cause the balloon material to flow. As a result, the imprint of the stent is formed in the sleeve without disadvantageously affecting balloon performance characteristics such as rupture pressure, inflated shape, and compliance. Unlike the sleeve, the balloon under the sleeve preferably does not have an imprint of the stent, or at least does not have an imprint of the stent which remains in the balloon after the inflated balloon is deflated to radially collapse the balloon and sleeve away from the expanded stent.

Flowing the sleeve material into the stent gaps causes localized thinning of the wall thickness of the sleeve, unlike a stent delivery catheter in which the balloon material maintains a constant wall thickness and deforms around the edges of the stent as the stent is mounted thereon. This localized thinning in the sleeve does not disadvantageously affect performance of the catheter. In contrast, if the balloon material was caused to flow, the localized thinning would result in a reduction of the balloon rupture pressure. Additionally, in an embodiment having a therapeutic agent such as a drug delivery coating on the stent, the low glass transition temperature of the sleeve facilitates securely mounting the stent without exposing the therapeutic agent to high temperatures and/or pressures which can damage the agent or its matrix during stent mounting.

By flowing the elastomeric material, a permanent imprint is formed. Additionally, the imprint can be made flush with the outer surface of the stent (e.g., so that at least part of the portions of the sleeve protruding between the adjacent stent struts have an outer diameter equal to an outer diameter of the nonexpanded stent), to maximize the mechanical interference between the stent and sleeve. In contrast, if the material underlying the stent is not caused to reflow, as for example if the stent is gently crimped without flowing the underlying material, the imprint is not formed and at most only a temporary and minor mechanical interference is produced between the stent and underlying material. Additionally, because the elastomeric material of the sleeve is caused to flow, it penetrates within very small gaps of densely collapsed stent struts. In contrast, if the material is not caused to reflow, the material does not expand into such small stent gaps, or at least not without damaging the material. Thus, the stent can be collapsed to a greater degree, providing a smaller profile for introduction and advancement within the body lumen.

The sleeve preferably exerts a radially compressive force on the balloon when it is expanded. As a result, when the inflation pressure inside the balloon is released the sleeve acts to compress the balloon and push the inflation fluid out of the balloon, speeding up the deflation time. Similarly, the sleeve preferably improves balloon rewrap due to the radially compressive force on the wings of the deflated balloon, forcing the wings to a lower profile. The sleeve preferably radially collapses away from the expanded stent without resistance, and the deflated catheter balloon is withdrawn from the expanded stent without snagging on the stent.

One aspect of the invention is directed to a method of mounting a stent on the stent delivery balloon catheter having the elastomeric sleeve between the stent and balloon. To releaseably mount the stent onto the sleeved balloon catheter, the stent is radially compressed onto the outer surface of the sleeve, and the balloon is then partially inflated to radially expand the balloon and sleeve, so that the sleeve extends into the gaps in the stent wall between adjacent stent struts. The stent is restrained from radially expanding by a radial restraining member around an outer surface of the stent, so that the sleeve can be forced into the stent gaps using inflation pressures higher than those which normally cause radial expansion of the stent. The sleeve is softened, as for example using heat or solvent, which facilitates the flowing of the sleeve material within the stent gaps of the radially restrained stent. As a result, the stent gaps are completely or at least partially filled by portions of the sleeve protruding between the adjacent stent struts, and an imprint of the stent is permanently formed in the outer surface of the sleeve. The balloon interior is then depressurized, leaving the stent within the imprint and embedded in the sleeve on the noninflated balloon.

By mounting a stent on the elastomeric sleeve in accordance with a method of the invention, the sleeve improves the mechanical interference between the stent and the delivery system. The sleeve is caused to flow in order to encapsulate the inside surface and typically at least in part the sides of the stent, and protrude within the stent wall, resulting in changes in the wall thickness of the sleeve. However, the sleeve reflows at sufficiently low temperatures and pressures during stent mounting so that the balloon material and/or therapeutic agent are not damaged during the stent mounting. These and other advantages of the invention will become more apparent from the following detailed description and exemplary drawings.

### BRIEF DESCRIPTION OF THE DRAWINGS

FIG. 1 is an elevational view of a stent delivery balloon catheter embodying features of the invention, illustrating the distal end of the catheter partially in section.

FIG. 1A is an elevational view of the distal end of the catheter of FIG. 1, with the sleeve illustrated partially in section.

FIGS. 2-3 are transverse cross sectional views of the stent delivery balloon catheter of FIG. 1, taken along lines 2-2, and 3-3, respectively.

FIG. 3A illustrates an alternative embodiment, in which the imprint in the sleeve is shallower.

FIG. 4 illustrates the stent delivery balloon catheter of FIG. 1 with the balloon in an inflated configuration.

FIG. 4A is an enlarged view of the catheter of FIG. 4, taken within circle 4A.

FIGS. 5 and 5A are transverse cross sectional views of the stent delivery balloon catheter of FIG. 4, taken along lines 5-5 and 5A-5A, respectively.

FIG. 6 illustrates the stent delivery balloon catheter of FIG. 1 with the balloon in a deflated configuration radially collapsed from the expanded stent.

FIG. 7 is a transverse cross sectional view of the stent delivery balloon catheter of FIG. 6, taken along line 7-7.

### DETAILED DESCRIPTION OF THE PREFERRED EMBODIMENTS

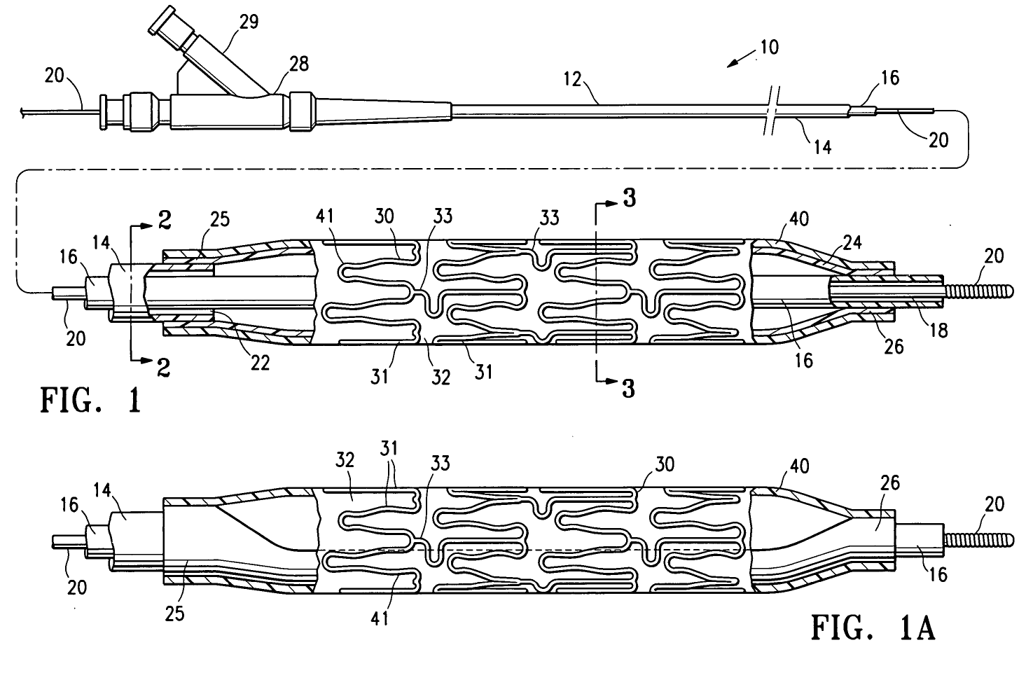

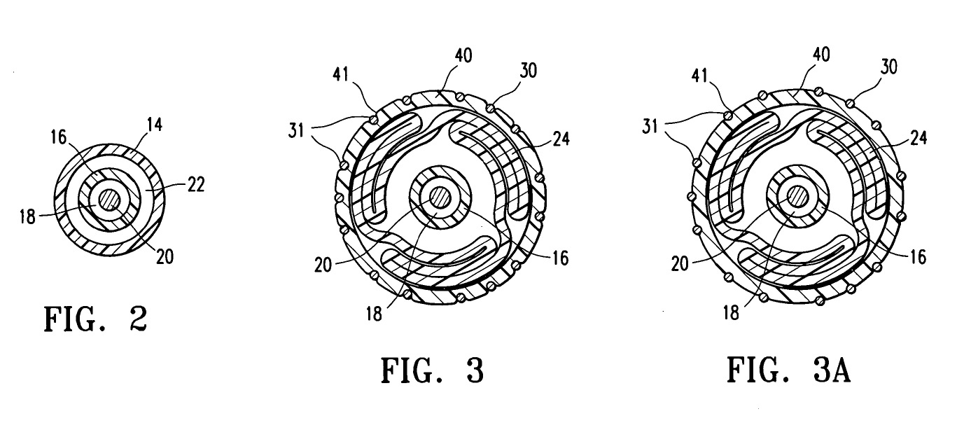

FIG. 1 illustrates an over-the-wire type stent delivery balloon catheter 10 embodying features of the invention. Catheter 10 generally comprises an elongated catheter shaft 12, an inflatable balloon 24 on a distal shaft section, an elastomeric sleeve 40 on the balloon 24, and a stent 30 mounted on the sleeve 40. In the illustrated embodiment, the shaft comprises an outer tubular member 14 defining an inflation lumen 22 therein, and an inner tubular member 16 defining a guidewire lumen 18 therein configured to slidingly receive a guidewire 20. Specifically, in the illustrated embodiment, the coaxial relationship between outer tubular member 14 and inner tubular member 16 defines annular inflation lumen 22, as best shown in FIG. 2 illustrating a transverse cross section of the distal end of the catheter shown in FIG. 1, taken along line 2-2. In the embodiment illustrated in FIG. 1, the guidewire lumen 18 extends to the proximal end of the catheter. Inflatable balloon 24 has a proximal skirt section 25 sealingly secured to the distal end of outer tubular member 14 and a distal skirt section 26 sealingly secured to the distal end of inner tubular member 16, so that the balloon interior is in fluid communication with inflation lumen 22. An adapter 28 at the proximal end of catheter shaft 12 is configured to provide access to guidewire lumen 18, and to direct inflation fluid through arm 29 into inflation lumen 22.

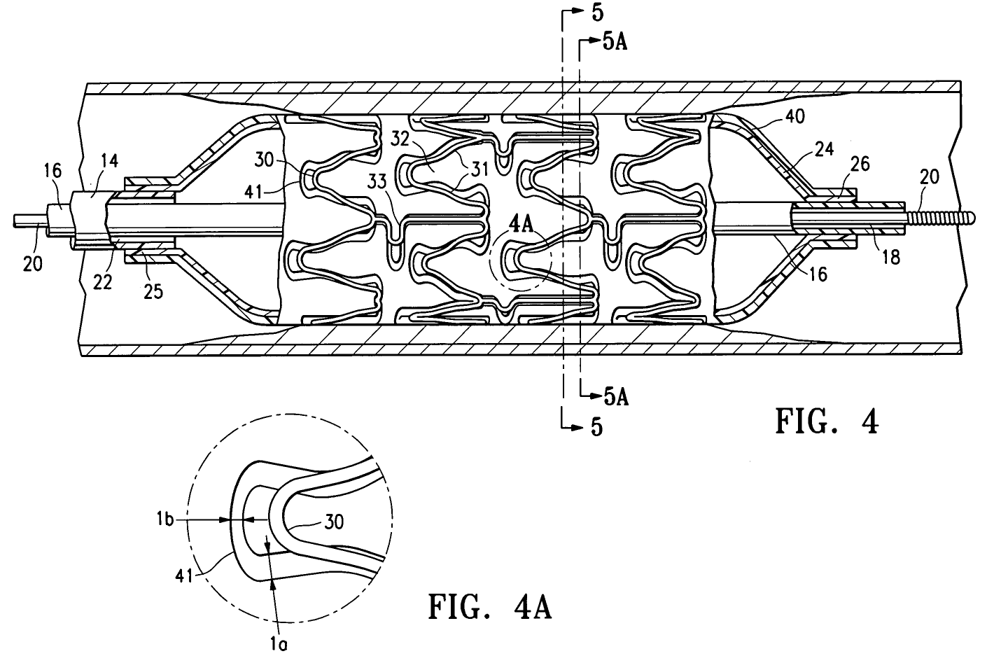

FIG. 1 illustrates the balloon 24, in a folded, noninflated configuration with wings (see FIGS. 1A and 3) wrapped around the circumference of the balloon prior to inflation of the balloon, and with a sleeve 40 encircling the wings of the folded, noninflated balloon. FIG. 1A illustrates the sleeve 40 in a partial sectional view, and the balloon 24 not in a partial sectional view, to show a wing of balloon 24 underneath the sleeve 40. The balloon 24 typically has two or more, and most preferably three wings in the noninflated configuration. For ease of illustration, a substantial gap is illustrated between the inner surface of the inflatable balloon interior and the shaft inner tubular member 16 in FIGS. 1 and 3, although it should be understood that the noninflated balloon is typically collapsed down around to inner tubular member in the noninflated configuration. The balloon inflates to a generally cylindrical configuration (see FIG. 4) with a central, working length inflated section, a proximal inflated conical section proximal to the stent (and distal to the proximal skirt section 25), and a distal inflated conical section distal to the stent (and proximal to the distal skirt section 26). Stent 30 is in a nonexpanded configuration extending along the central, working length section of the balloon 24 in FIG. 1. The distal end of catheter 10 may be advanced to a desired region of the patient's body lumen in a conventional manner with the balloon in the noninflated configuration, and the balloon 24 inflated by directing inflation fluid into the balloon interior to expand the stent 30. The balloon 24 is then deflated, leaving the stent 30 implanted in the body lumen.

The stent 30 generally comprises an open-walled body of interconnected, spaced-apart stent struts 31 with gaps 32 between adjacent stent struts. In the illustrated embodiment, the stent struts 31 form rings which have a serpentine wave pattern of opposed turns and which are longitudinally spaced apart and connected by links 33. However, the stent 30 can have a variety of suitable configurations as are conventionally known. Although not illustrated, the stent 30 typically carries a therapeutic agent (e.g., a stent commonly referred to as a “drug-eluting stent”). For example, in one preferred embodiment, the stent has a coating on a surface of the stent, and the coating has the therapeutic agent and optionally other substances such as a polymeric matrix. The terminology therapeutic agent should be broadly understood to include a wide variety of agents with therapeutic and/or prophylactic effects, as are conventionally known, including for example drugs for reducing restenosis.

The sleeve 40 is a solid-walled tube, which in the illustrated embodiment has a length equal to the length of the balloon 24. Alternatively, the sleeve can have a length greater than or less than the length of the balloon. 24. The sleeve wall thickness is generally about 0.013 to about 0.13 millimeters (0.0005 to about 0.005 inches), and more preferably about 0.038 to about 0.076 millimeters (0.0015 to about 0.0030 inches). One or both of the end sections of the sleeve 40 are typically bonded to the balloon. In a presently preferred embodiment, at least the sleeve central section extending along the balloon central, working length section (underneath the stent) is not bonded or otherwise adhered to the balloon. In one embodiment, only the proximal and distal end sections of the sleeve extending along the proximal and distal skirt sections 25, 26 of the balloon 24 are bonded to the balloon. In an alternative embodiment, the distal end section or the proximal end section of the sleeve is open, e.g., not bonded to the balloon or the catheter shaft. The sleeve 40 has a proximal expandable section proximal to the stent, and a distal expandable section distal to the stent.

In the noninflated configuration illustrated in FIG. 1, the stent 30 sits within an imprint 41 of the stent in the outer surface of the sleeve 40. The imprint 41 is visible in the sleeve in the noninflated, inflated, and deflated configurations, as discussed in more detail below. The imprint 41 results in portions of the sleeve 40 protruding between the adjacent stent struts 31. The elastomeric material of the sleeve has flowed around the stent and into contact with the side surface of the stent, so that the protruding portions of the sleeve fully encapsulate the side surfaces of the stent struts 31, as best illustrated in FIG. 3 showing a transverse cross section of the balloon of FIG. 1 taken along line 3-3. In an alternative embodiment, the protruding portions of the sleeve forced within stent do not encapsulate, or only partially encapsulate, the side surfaces of the stent. As a result of the reflowing of the sleeve 40, the wall thickness of the portion of the sleeve 40 underneath the stent struts 31 is thinner than the wall thickness of the protruding portion of the sleeve 40 between the stent struts 31.

In the embodiment illustrated in FIG. 3, the side surfaces of the stent are fully encapsulated and all of the portions of the sleeve protruding between the adjacent stent struts have an outer diameter equal to an outer diameter of the nonexpanded stent. However, a different degree of imprinting (i.e., the extent of encapsulation of the stent) and level of protrusion (i.e., the depth of the imprint formed in the sleeve) can be produced, depending on the elevated temperature, duration, and/or elevated pressure used during the stent mounting. For example, in an alternative embodiment, the protruding portions of the sleeve have an outer diameter greater than the inner diameter of the nonexpanded stent but less than the outer diameter of the nonexpanded stent (see e.g., FIG. 3A). The degree of imprinting/level of protrusion affects the failure mode in the event of stent dislodgement. For example, with a small protrusion level (e.g., less than the outer diameter of the nonexpanded stent), if the nonexpanded stent does dislodge from the sleeve, it typically slides relative to the sleeve, without damage to the stent or movement of the sleeve relative to the balloon. In contrast, with a large protrusion level, dislodgement of the nonexpanded stent, albeit less likely, can damage the stent and/or cause the sleeve to shift (e.g., stretch and/or buckle) relative to the balloon. Ideally, dislodgement of the stent is highly unlikely, but occurs if at all without damage to the stent or stent delivery catheter. Thus, the degree of imprinting/protrusion level can thus be tailored to achieve the desired performance characteristics. In one embodiment (not illustrated), a part of the protruding portions of the sleeve have an outer diameter equal to the outer diameter of the nonexpanded stent mounted on the balloon, but the degree of imprinting is less than that illustrated in the embodiment of FIG. 3, such that the sleeve does not fully encapsulate the side surfaces of the stent (i.e., the conditions used during stent mounting are such that the elastomeric material of the sleeve is not fluid enough during stent mounting to fully encapsulate the side surfaces of the stent)

FIG. 4 illustrates the balloon in the inflated configuration, expanded to the balloon nominal outer diameter so that the stent is expanded against the stenosed region of the body lumen wall. Although in one embodiment the sleeve does exert a radially compressive force on the balloon, in a preferred embodiment, the sleeve at most only minimally constrains the balloon from inflating, and thus does not disadvantageously prevent or inhibit the balloon from inflating. In a presently preferred embodiment, a lubricant is not provided between the sleeve and balloon. As a result, sliding of the sleeve on the balloon is prevented or minimized. A lubricant would cause the sleeve to slide on the balloon, taking the stent with it and thus interfering with the accurate implantation of the stent in the patient's body lumen.

As the sleeve radially expands and stretches, the imprint 41 in the sleeve is stretched radially but typically is not significantly stretched axially (i.e., longitudinally). Thus, as best illustrated in FIG. 4A, showing an enlarged view of the catheter of FIG. 4 taken within circle 4A, the width of the imprint at 1a is wider than the stent strut width, but at the peak of the turn of the stent ring at 1b the width of the imprint remains the same as the stent strut width.

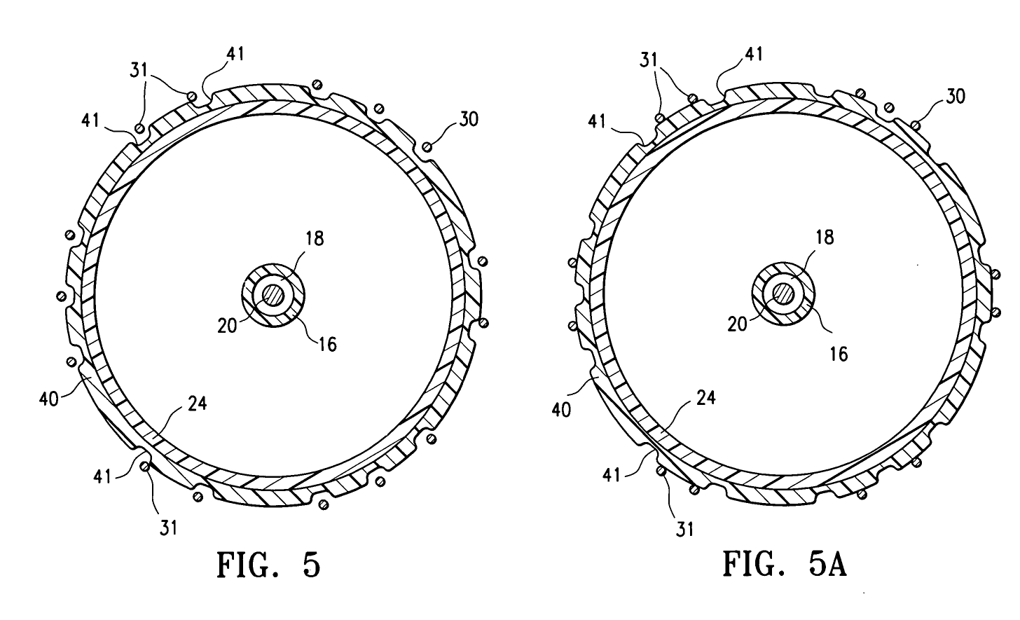

As the stent radially expands to the expanded configuration illustrated in FIG. 4, the stent rings shorten axially and the turns of each ring open-up. Depending on the degree to which the stent deforms as it radially expands, the expanded stent typically has a shape which no longer matches the imprint of the noninflated stent formed in the sleeve. Although the imprint stretches somewhat to accommodate the stent strut as the balloon is inflated, the stretching of the imprint is limited. As a result, at some point during the inflation of the balloon the stent no longer fits within the imprint, so that the expanded stent is in whole or in part outside the imprint. A stent which did not deform as it radially expands would typically remain within the imprint in the expanded configuration and until the inflated balloon was deflated to radially collapse the balloon and sleeve away from the expanded stent. FIGS. 5 and 5A illustrate a transverse cross sections of the inflated balloon of FIG. 4, taken along lines 5-5 and 5A-5A, respectively.

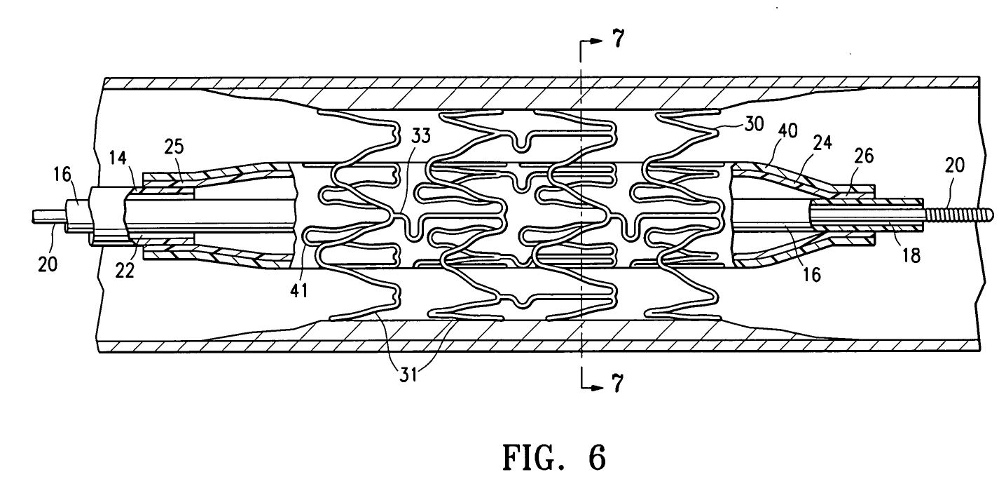

FIG. 6 illustrates the balloon 24 in the deflated configuration, with the balloon 24 and sleeve 40 radially collapsed away from the expanded stent 30. The imprint 41 remains clearly visible in the deflated sleeve 40.

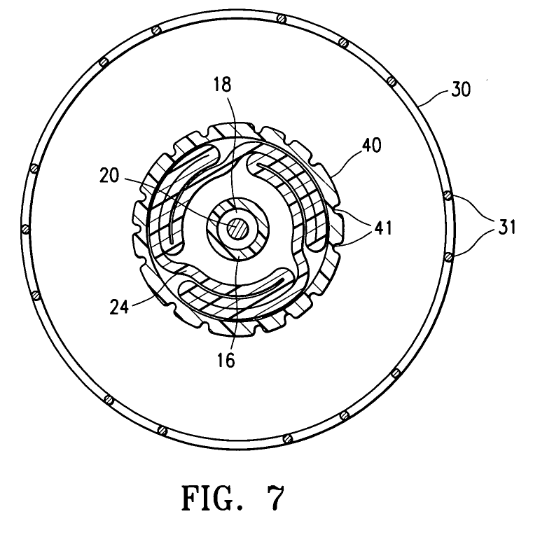

As best illustrated in FIG. 7, showing a transverse cross section of the balloon of FIG. 6, taken along line 7-7, the inflated balloon deflates to deflated configuration, with the sleeve encircling the reformed wings of the deflated balloon. Although illustrated in FIG. 7 with the balloon wings rewrapped around a circumference of the balloon for ease of illustration, it should be understood that the reformed wings of the deflated balloon don't necessarily rewrap. For example, the sleeve 40 bends the wings as the balloon deflates so that they lay close to the inner member, reducing the profile of the system, with some of the reformed wings rewrapping around the balloon and some buckling against the inner member.

In a method of mounting the stent 30 on the balloon catheter 10 to form the stent delivery catheter of FIG. 1, the stent 30 is positioned on the balloon catheter 10 so that the stent is on an outer surface of the elastomeric sleeve 40. A radially compressive force is applied on an outer surface of the stent, thereby decreasing the outer diameter of the stent on the balloon catheter. For example, in one embodiment, the stent is crimped onto the outer surface of the sleeve 40, and then an inelastic sheath is placed on the outer surface of the stent, and the stent is pressed to radially collapse the stent down onto the sleeve. The method further includes gripping the stent in the sleeve by softening the sleeve and introducing inflation media into the interior of the balloon to radially expand the balloon and softened sleeve with the stent restrained from radially expanding by a radial restraining member around an outer surface of the stent, so that the softened sleeve flows within the gaps of the radially restrained stent. The radial restraining member is typically a mold having an inner chamber configured to receive the balloon portion of the catheter 10, with the sleeve 40 and stent 30 on the balloon 24. The balloon interior is pressurized at typically about 50 to about 300 psi, preferably about 75 to about 200 psi, during the stent gripping.

In one embodiment, softening the sleeve comprises heating the sleeve. The sleeve can be heated by a variety of suitable methods. In a presently preferred embodiment, the entire balloon catheter is placed in an oven to heat the sleeve. Although the entire balloon catheter is heated in the oven, the elevated temperature (above room temperature) of the oven is greater than the glass transition temperature of the elastomeric material of the sleeve but not greater than, and preferably less than the glass transition temperature of a polymeric material of the balloon, and significantly less than a thermal limit of the therapeutic agent (e.g., about 35° C. less than the thermal limit of the therapeutic agent). As a result, the balloon and therapeutic agent are not disadvantageously affected by being heated to a temperature which is above the glass transition temperature of the sleeve. For example, in one embodiment, a sleeve formed of a blend of 75% TECOFLEX 80A (polyurethane)/25% VECTOR 7400 (styrenic block copolymer) polymeric material (which will reflow during stent mounting at temperatures less than approximately 55° C.) is heated at about 30° C. to about 45° C. to soften the sleeve, on a balloon formed of polyether block amide (PEBAX) polymeric material having a glass transition temperature of about 45° C. The thermal limit of the therapeutic agent will depend on the therapeutic agent and the delivery matrix employed to deliver the therapeutic agent, but it typically significantly higher than the temperatures required to reflow the sleeve material during stent mounting. For example, in one embodiment, the thermal limit of a therapeutic agent coating on the stent is about 80° C. The thermal limit of the therapeutic agent should be understood to include the elevated temperatures above which a matrix containing the therapeutic agent is damaged in addition to the temperatures above which the therapeutic agent itself is damaged (e.g., damage affecting release rate, therapeutic agent activity and concentration, etc.).

In an alternative embodiment, softening the sleeve comprises exposing the sleeve to a plasticizing solvent. The sleeve is exposed to the plasticizing solvent by directly applying the liquid solvent using a variety of techniques including spraying, brushing, and dipping, or by vapor phase deposition. In a presently preferred embodiment, the plasticizing solvent is sprayed onto the sleeve. For example, sleeve materials such as Tecoflex and Vector Blends can be temporarily softened (i.e., plasticized without dissolving) with solvents such as isopropyl alcohol (IPA) or acetone, producing effects similar to heating. The solvent concentration is typically about 50% to about 100%. The affect of the plasticizing solvent is temporary, so that after drying, the treated material has the integrity and properties of the original material. The plasticizing solvent is used without heating the sleeve, or alternatively, together with relatively low temperature heating (e.g., about 25 to about 35° C.). Thus, the softened sleeve can be heated to further soften the sleeve during the stent gripping and/or to dry the plasticizing solvent on the sleeve. The presently preferred plasticizing solvents have a relatively high vapor pressure so that the solvent dries at relatively low temperatures (e.g., less than or equal to about 25° C.).

The pressurizing fluid is then removed from the balloon interior, and the inelastic sheath is removed from the stent. In a presently preferred embodiment, the inelastic sheath remains on the stent during the stent pressing and during the gripping of the stent (i.e., pressurization to flow the sleeve material within the stent gaps). In an alternative embodiment, a second inelastic sheath having a larger outer diameter than the pressed inelastic sheath may be used during the gripping of the stent, so that the balloon and stent radially open-up to a greater extent to facilitate subsequent inflation of the balloon.

The dimensions of catheter 10 are determined largely by the size of the balloon and guidewire to be employed, the catheter type, and the size of the artery or other body lumen through which the catheter must pass or the size of the stent being delivered. Typically, the outer tubular member 14 has an outer diameter of about 0.025 to about 0.04 inch (0.064 to 0.10 cm), usually about 0.037 inch (0.094 cm), and the wall thickness of the outer tubular member 14 can vary from about 0.002 to about 0.008 inch (0.0051 to 0.02 cm), typically about 0.003 to 0.005 inch (0.0076 to 0.013 cm). The inner tubular member 16 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). The overall length of the catheter 10 may range from about 100 to about 150 cm, and is typically about 143 cm. Preferably, balloon 24 has a length about 0.8 cm to about 6 cm, and an inflated working diameter of about 2 mm to about 10 mm.

Inner tubular member 16 and outer tubular member 14 can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives. Although the shaft is illustrated as having an inner and outer tubular member, a variety of suitable shaft configurations may be used including a dual lumen extruded shaft having a side-by-side lumens extruded therein. Similarly, although the embodiment illustrated in FIG. 1 is an over-the-wire type balloon catheter, the catheter of this invention may comprise a variety of intravascular catheters, such as rapid exchange type balloon catheters. Rapid exchange catheters generally comprise a shaft having a relatively short guidewire lumen extending from a guidewire distal port at the catheter distal end to a guidewire proximal port spaced a relatively short distance from the distal end of the catheter and a relatively large distance from the proximal end of the catheter.

While the present invention is described herein in terms of certain preferred embodiments, those skilled in the art will recognize that various modifications and improvements may be made to the invention without departing from the scope thereof. Although illustrated on a balloon having wings wrapped around the balloon in the noninflated configuration, the elastomeric sleeve can alternatively be on a wingless balloon in an alternative embodiment. Similarly, although discussed primarily in terms of a drug delivery stent, the stent delivery system can alternatively have a stent without a drug (e.g., a “bare metal stent”). Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed:

1. A stent delivery balloon catheter, comprising
a) an elongated shaft having an inflation lumen and a guidewire lumen;
b) an inflatable balloon on a distal shaft section, having an interior in fluid communication with the inflation lumen;
c) an elastomeric sleeve on an outer surface of the balloon; and
d) an expandable stent which has a therapeutic agent and which is releaseably mounted on an outer surface of the sleeve, with an imprint of the stent in an outer surface of the sleeve in a noninflated configuration, an inflated configuration, and a deflated configuration after the inflated balloon is deflated to radially collapse the balloon and sleeve away from the expanded stent.

2. The balloon catheter of claim 1 wherein the stent is within the imprint in the noninflated configuration.

3. The balloon catheter of claim 1 wherein the stent comprises an open-walled body of stent struts with gaps between adjacent stent struts, and portions of the sleeve protruding between the adjacent stent struts are in contact with side surfaces of the struts.

4. The balloon catheter of claim 3 wherein at least part of the portions of the sleeve protruding between the adjacent stent struts have an outer diameter equal to an outer diameter of the nonexpanded stent.

5. The balloon catheter of claim 3 wherein at least part of the portions of the sleeve protruding between the adjacent stent struts have an outer diameter larger than an inner diameter of the nonexpanded stent and smaller than an outer diameter of the nonexpanded stent.

6. The balloon catheter of claim 1 wherein the noninflated configuration of the balloon is a folded, noninflated configuration with wings wrapped around the circumference of the balloon prior to inflation of the balloon, so that the sleeve encircles the wings of the folded, noninflated balloon.

7. The balloon catheter of claim 1 wherein the sleeve has a tubular body which radially expands from a nonfolded noninflated configuration without unfolding.

8. The balloon catheter of claim 1 wherein the therapeutic agent is a coating on a surface of the stent.

9. The balloon catheter of claim 1 wherein the balloon does not have an imprint of the stent in an outer surface of the balloon.

10. The balloon catheter of claim 1 wherein the sleeve has a proximal end section and a distal end section adjacent the expandable section of the sleeve, and at least one of the proximal and distal end sections are bonded to an outer surface of the catheter shaft or balloon, and the expandable section of the sleeve is not bonded to the balloon.

11. A stent delivery balloon catheter, comprising
a) an elongated shaft having an inflation lumen and a guidewire lumen;
b) an inflatable balloon on a distal shaft section, having a proximal and a distal section sealingly secured to the shaft so that an interior of the balloon is in fluid communication with the inflation lumen, and having an inflatable section;
c) an elastomeric sleeve on an outer surface of the balloon, having an inner surface in direct contact with the balloon without a lubricant between the sleeve and balloon, and the inner surface of the sleeve having at least a section thereof which extends along the inflatable section of the balloon and which is not bonded to the balloon; and
d) an expandable stent which has a therapeutic agent and which is releaseably mounted on an outer surface of the sleeve, with an imprint of the stent in an outer surface of the sleeve in a noninflated configuration, an inflated configuration, and a deflated configuration after the inflated balloon is deflated to radially collapse the balloon and sleeve away from the expanded stent.

12. A method of mounting a stent on a stent delivery balloon catheter, comprising:
a) positioning a stent on a balloon catheter, the balloon catheter having an elongated shaft with an inflation lumen and a guidewire lumen, an inflatable balloon on a distal shaft section with an interior in fluid communication with the inflation lumen, and an elastomeric sleeve on an outer surface of the balloon, so that the stent is on an outer surface of the elastomeric sleeve, the stent having a therapeutic agent and an open-walled body of stent struts with gaps between adjacent stent struts;
b) applying a radially compressive force on an outer surface of the stent and thereby decreasing the outer diameter of the stent on the balloon catheter;
c) softening the sleeve and introducing inflation media into the interior of the balloon to radially expand the balloon and softened sleeve with the stent restrained from radially expanding by a radial restraining member around an outer surface of the stent, so that the softened sleeve flows within the gaps of the radially restrained stent, to form an imprint of the stent in the outer surface of the sleeve; and
d) removing the inflation media from the interior of the balloon.

13. The method of claim 12, wherein softening the sleeve comprises heating the sleeve.

14. The method of claim 13 wherein heating the sleeve comprises placing the entire balloon catheter in an oven, so that the entire balloon catheter is heated.

15. The method of claim 13 wherein the sleeve is heated to an elevated temperature above the glass transition temperature of the elastomeric material of the sleeve, and less than or equal to the glass transition temperature of a polymeric material of the balloon, and significantly less than a thermal limit of the therapeutic agent.

16. The method of claim 15 wherein the sleeve is heated to about 25 to about 50° C.

17. The method of claim 12, wherein softening the sleeve comprises exposing the sleeve to a solvent.

18. The method of claim 12 including a placing an inelastic sheath on the outer surface of the stent which remains on the stent during b) and c), and removing the inelastic sheath after d).

19. The method of claim 18 including, before b), crimping the stent down onto the outer surface of the sleeve.
